(19) United States
(12) Patent Application Publication
(10) Pub. No.: US 2006/0263449 A1
(43) Pub. Date: Nov. 23, 2006

(54) Herbal composition for treatment of arthritic disorders, skin inflammatory disorders and pain

(75) Inventors: Li-Wei Hsu, Taichung City (TW); Su-Chen Chang, Taichung City (TW); Kuo-Sheng Chuang, Jian Township (TW); Hsuan-Yi Lin, Taichung City (TW); Jyh-chong Chen, Taichung City (TW)

Correspondence Address: Scully, Scott, Murphy & Presser, 400 Garden City Plaza, Garden City, NY, 11530-3319, US

(73) Assignee: Advanced Gene Technology, Corp., Taiwan (CN)

(21) Appl. No.: 11/134,140

(22) Filed: May 20, 2005

(51) Int. Cl. A61K 36/185

(52) U.S. Cl. 424/725

(57) ABSTRACT

The present invention provides a method for treating arthritic disorders, skin inflammatory disorders and pain, comprising administering to a subject an extract of Andrographis paniculata Nees, and also provides a herbal composition used therefor that comprises the extract of Andrographis paniculata Nees.

### TECHNICAL FIELD OF THE INVENTION

The present invention relates to an herbal extract for the treatment of arthritic disorders, skin inflammatory disorders and pain. The herbal extract acts as a tumor necrosis factor-α (TNFα) antagonist. Specifically, the herb is Andrographis paniculata Nees.

### BACKGROUND OF THE INVENTION

Arthritic disorders include osteoarthritis (OA), rheumatoid arthritis (RA), juvenile arthritis, gout, ankylosing spondylitis and other joint disorders. All of arthritic disorders are characterized by inflammation and pain, and most of arthritic disorders are progressive. Neither the pathogenic causes that commence arthritic disorders, nor their mechanisms have been well identified.

Prostaglandins were recognized to cause inflammation and in turn to cause pain. It was found that cyclooxygenase-2 (Cox-2), which is an isoform of cyclooxygenase-1 (Cox-1) and is inducibly expressed in response to a variety of proinflammatory stimuli, triggers the release of prostaglandins. COX-2 inhibitors were deemed as a potent drug for treating inflammation and pain. Two synthetic COX-2 inhibitors, i.e. celecoxib (Celebrex) and rofecoxib (Vioxx), were commercially available in the market for treating arthritis and pain. However, it has been demonstrated that all of the celecoxib (Celebrex) and rofecoxib (Vioxx) and the other two COX-2 inhibitors (i.e. lumiracoxib and etoricoxib) that had not yet been approved by the Food and Drug Administration (FDA) showed cardiovascular toxicity. There is not a drug for treating arthritis and pain, while showing low toxicity and low side effects. TNFα is now recognized as a multifunctional cytokine that plays key roles in acute and chronic inflammation, anti-tumor response and infection. Enhanced TNFα synthesis is associated with the development of RA, psoriatic arthritis and inflammatory bowel disease. Based on clinical studies, it has been confirmed that TNFα is central in the pathogenesis of RA. A TNFα antagonist has been used for treating inflammatory diseases; for example, etanercept (Enbrel) has been used for treating RA, and infliximab (Remicade) has been used for treating Crohn's disease and RA. An anti-TNFα drug, adalimumab (Humira; Abbott), has been also commercially available. In addition, D2E7 (a human anti-TNFα monoclonal antibody, Knoll Pharmaceuticals) is being developed for treating Crohn's disease and RA. CDP 571 (Celltech) is being developed for treating Crohn's disease and CDP 870 (Celltech) is being developed for treating RA.

It was found, however, that etanercept and infliximab showed a potential risk of worsening congestive heart failure, and adalimumab showed an increased potential in the development of lymphoma.

Though several small-molecule TNFα synthesis inhibitors including thalidomide (Celgene), p38 kinase inhibitors and TACE inhibitors are currently tested in clinical trial (see Nature Reviews, Drug Discovery, September 2003, Vol. 2, pages 736-746), however they are not TNFα inhibitors at all.

It was recited in Chinese herb books that andrographolide, an active ingredient isolated from Andrographis paniculata Nees, has a function on inhibiting bacterial infection and on the treatment of airway and lung inflammation. Andrographolide was different in chemical structure from those small-molecule TNFα synthesis inhibitors as described above. The report “Phytomedicine, 2002 October, 9(7): 598-605” described that both andrographolide and Kan Jang increase the formation of TNFα and β2MG in the cultivated, phytohemagglutinin (PHA)-stimulated whole blood cell. In addition, there was no disclosure in literatures that andrographolide or even Andrographis paniculata Nees per se could be used for treating arthritic disorders, especially treating arthritis.

### SUMMARY OF THE INVENTION

The present invention discloses a method for treating arthritic disorders, skin inflammatory disorders and pain, comprising administering to a subject an extract of Andrographis paniculata Nees. In a preferred embodiment, the arthritic disorder is arthritis (especially RA and OA) and neuralgia induced by ankylosing spondylitis, and the skin inflammatory disorders are psoriasis, eczema and dermatitis. In another preferred embodiment, the administration mode is oral administration.

The present invention discloses an herbal composition for treating arthritic disorders, skin inflammatory disorders and pain, comprising an extract of Andrographis paniculata Nees and a pharmaceutically acceptable excipient. When administered to a subject, the herbal composition shows low toxicity and low side effects, compared to the well-known COX-2 inhibitors and TNFα inhibitors.

In another aspect, the present invention discloses the use of an extract of Andrographis paniculata Nees as a TNFα antagonist.

### BRIEF DESCRIPTION OF THE DRAWINGS

FIG. 1 shows a typical HPLC (High Performance Liquid Chromatography; TSK Gel ODS 80™ (TOSOH) reversed phase column (250×4.6 mm) filled with 5 μm gel particle) chromatogram for quality control of the samples of Andrographis paniculata Nees, wherein the peak of andrographolide (AG) is indicated.

FIG. 2 shows a process flowchart for the preparation of the herbal composition of the present invention.

FIG. 3 shows the in vitro TNFα inhibitory activity of the herbal composition of the present invention and its cytotoxicity on L929 cell proliferation/cytotoxicity assay.

FIG. 4 shows an improvement on symptom relief in 32 patients suffering from RA, OA or neuralgia after 7-day continual administration of the herbal composition of the present invention.

FIG. 5 shows an improvement on symptom relief in 32 patients suffering from RA, OA or neuralgia after 14-day continual administration of the herbal composition of the present invention.

FIG. 6 shows improvement % in 32 patients suffering from RA, OA or neuralgia respectively.

### DETAILED DESCRIPTION OF THE INVENTION

Pharmaceuticals commercially available in the market for treating arthritic disorders and pain include non-steroidal anti-inflammatory drugs (NSAIDs) and corticosteroids, wherein the NSAIDs are the most commonly used drugs. However, the use of the NSAIDs in therapy is limited because of their propensity to causing the undesired gastrointeninal (GI) injury. Corticosteroids may also cause multiple side effects and are often not effective.

For treating arthritic disorders and pain, new biopharmaceuticals have been developed, which showed to offer a dramatic clinical benefit in therapy. These biopharmaceuticals block or antagonize the biological action of a specific cytokine. These cytokines include members of the interleukins and tumor necrosis factors.

Tumor necrosis factor α (TNFα) is a well-known proinflammatory cytokine with a wide range of biological functions. It was known that TNFα affects the growth, differentiation, survival and physiological functions of a variety of cells. TNFα interacts with two distinct cell-surface receptors: TNFR1(p55) and TNFR2(p75), both of which receptors bind to TNFα with high affinity. TNFα is intimately involved in the nervous system and inflammation disorders, and plays a central role in the response to virally induced or disease-induced injury. TNFα is also central in neuronal apoptosis.

Specific inhibitors of TNFα provide the possibility of therapeutic intervention in TNFα-mediated disorders. These TNFα inhibitors were developed for treating systemic illnesses. For example, Immunex Corporation developed etanercept (Enbrel) for treating rheumatoid arthritis (RA); Johnson and Johnson developed infliximab (Remicade) for treating Crohn's Disease and RA; D2E7 (a human anti-TNFα monoclonal antibody; Knoll Pharmaceuticals) is being developed for treating RA and Crohn's Disease; and Celltech is developing CDP 571 for treating Crohn's Disease and CDP 870 for treating RA. The success in developing a TNFα antagonist for therapy served a positive result for the treatment of TNFα-associated diseases.

Despite the above therapeutic success, the use of a biological TNFα antagonist for therapy has limitations: (1) a potential to increase the undesired susceptibility to infection, especially for the patients afflicted with latent tuberculosis, (2) a limited response rate and (3) high cost.

For arthritic disorders, an improved treatment should not require the consumption of over-the-counter or prescription drugs. Herb medicines were always a candidate.

Herb medicines have been used for treating various diseases or disorders in human and animals. Herbal medications could be formulated to a variety of forms, including capsule, tablet or coated tablet; pellet; extract or tincture; powder; fresh or dried plant or plant part; tea; juice; cream and ointment; oil; or, as any combination thereof. Herbal medicines could be administered by any of conventional routes, including orally, rectally, parenterally, enterally, transdermally, intravenously, via feeding tubes, and topically.

There exists a demand for anti-inflammation, pain-relief herb-based therapeutic for treating arthritic disorders and pain, which has low toxicity and low side effects. For example, there is a demand for developing a non-aspirin therapeutic for the treatment of fever, pain and inflammation disorders, e.g. RA and OA. Preferably, the therapeutic can be orally administered.

Andrographis paniculata Nees is an old herb medicine. Andrographolide has a mild effect against fever and inflammation and on the enhancement of non-specific immunological function.

Upon applying the herbal chip disclosed in U.S. Pat. No. 6,645,719, one of the applicant's U.S. patents, for screening for an active ingredient of herbs that binds to TNFα, the inventors of the present invention found that there is an active ingredient contained in an extract of Andrographis paniculata Nees, which can specifically bind to TNFα and can inhibit the activity of TNFα. Further, upon in vitro and in vivo pharmacological assays, the inventors of the present invention surprisingly found that the extract of Andrographis paniculata Nees could be used for treating arthritic disorders and pain with low toxicity and low side effects, when compared to the well-known COX-2 inhibitors and TNFα inhibitors.

Further, the inventors of the present invention found that the extract of Andrographis paniculata Nees could be used for the treatment of skin inflammatory disorders, such as psoriasis, eczema and dermatitis.

The present invention therefore discloses an herbal composition for treating arthritic disorders, skin inflammatory disorders and pain, comprising an extract of Andrographis paniculata Nees and a pharmaceutically acceptable excipient.

For formulating the herbal composition, the extract of Andrographis paniculata Nees may be mixed with either a suitable powder base to form a dry powder, wherein the powder base is selected from the group of lactose, starch, starch derivatives such as multidextran, hydroxypropylmethyl cellulose and polyvinylpyrrolidone (PVP), or a suitable pharmaceutically acceptable liquid carrier, such as saline and glucose solution.

Pharmacologically acceptable additives, such as binders, sweeteners, thickeners, flavoring agents, disintegrating agents, coating agents, preservatives, lubricants and/or time delay agents may be added to the herbal composition. Preferably, suitable sweeteners include sucrose, fructose, glucose, aspartame and saccharin; suitable disintegrating agents include corn starch, methylcellulose, polyvinylpyrrolidine, xanthan gum, bentonite, alginic acid and agar; suitable flavoring agents include peppermint oil, oil of wintergreen, cherry, orange and raspberry flavoring; suitable coating agents include polymers or copolymers of acrylic acid and/or methacrylic acid and/or their esters, waxes, fatty alcohols, zein, shellac and glutan; suitable preservatives include sodium benzoate, vitamin E, alpha-tocopherol, ascorbic acid, methyl paraben, propyl paraben and sodium bisulphate; suitable lubricants include magnesium stearate, stearic acid, sodium oleate, sodium chloride and talc; and, suitable time delay agents include glyceryl monostearate and glyceryl distearate.

The present invention also discloses a method for treating arthritic disorders, skin inflammatory disorders and pain, comprising administering to a subject an extract of Andrographis paniculata Nees. In a preferred embodiment, the arthritic disorder is arthritis, especially RA and OA, and neuralgia induced by ankylosing spondylitis, and the skin inflammatory disorders are psoriasis, eczema and dermatitis. In another preferred embodiment, the administration mode includes oral, intravenous, parenteral and transdermal administration.

For oral administration, the herbal composition may be formulated to tablet, granule, powder, sachet, capsule, syrup, solution, suspension or emulsion. For intravenous and parenteral administration, the herbal composition may be formulated to pharmaceutically acceptable syrup, solution, suspension or emulsion. For transdermal administration, the herbal composition may be formulated to ointments, creams, lotions or patch.

The amount of the herbal composition to be administered for therapy is 300 to 3200 mg/day, preferably 400 to 2000 mg/day, depending upon the age, weight and physical conditions and severity of disorders of the patients. The herbal composition may be administered 1 to 4 times per day.

The following examples are used to further illustrate the technical content of the present invention. However, it should be noted that the present invention is not limited to the following examples.

### EXAMPLES

### Example 1

### Standardization Quality Control of the Herb

High Performance Liquid Chromatography (HPLC) was used to ensure the quality of the herb, Andrographis paniculata Nees, from batch to batch. The HPLC method and test result are described as follows:

(a) Preparation of an Herbal Extract for HPLC

Fifty gram of the herb were precisely weighted and placed in a 1000 ml sample bottle, to which 500 ml of distilled water were added. The resultant mixture was blended at room temperature under 1000 rpm for 5 min., and then heated at 100° C. for 2 hours. After filtering the mixture through a 60-mesh microfilter, 1 ml of the extract was taken to dilute with distilled water to a total volume of 50 ml. Prior to applying to HPLC, the 1 ml extract was filtered through a 0.45 μM membrane.

(b) HPLC Analysis

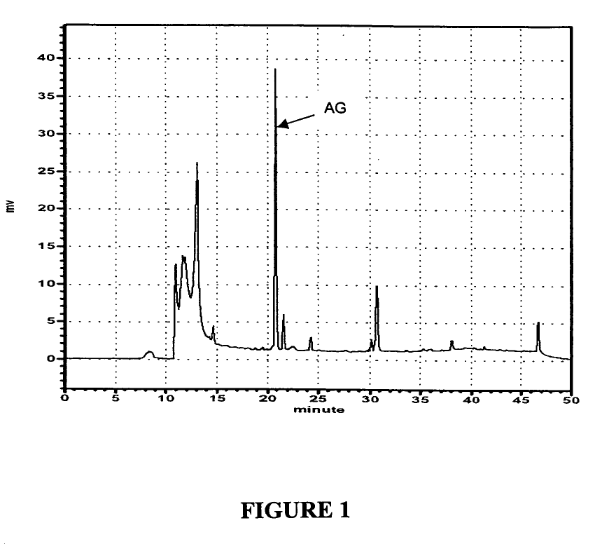

Separation procedure was performed on HPLC. 100 μl of the filtered supernatant, (i.e. the herb extract) was applied to a pre-equilibrated HPLC system (Shimadzu). TSK Gel 80™ reversed phase column (Tosoh) was used for separation at the wavelength of 230 nm. The solvent used for the separation were double distilled water and acetonitrile under 0 to 100% gradient for 96 minutes at a flow rate of 0.75 ml/min. The conditions are set as below and the obtained HPLC chromatogram is shown in FIG. 1.

| Time (minutes) | % of (H₂O) | % of (Acetonitrile) | Linearity |
|---|---|---|---|
| 0 | 100 | 0 | * |
| 5 | 70 | 30 | Linear |
| 25 | 50 | 50 | Linear |
| 30 | 70 | 30 | Linear |
| 35 | 100 | 0 | Linear |
| 45 | 100 | 0 | * |

The fractions obtained at the 20 to 21 minutes were further purified and then identified as andrographolide (AG) by LC/MS, HPLC and NMR spectrum. AG is used as a quality control standard for the herb material and bulk product.

The anti-TNFα activity of an extract of Andrographis paniculata Nees was demonstrated by the methods described in the following Example 3 and 4. The IC₅₀ value evaluated by in vitro L929 assay was 70 μM. The anti-inflammatory activity of the extract of Andrographis paniculata Nees that was studied in rats with collagen-induced arthritis was 40% decrease in edema of hint paws after a continual 7-day oral administration.

### Example 2

### Process for the Preparation of Herbal Composition

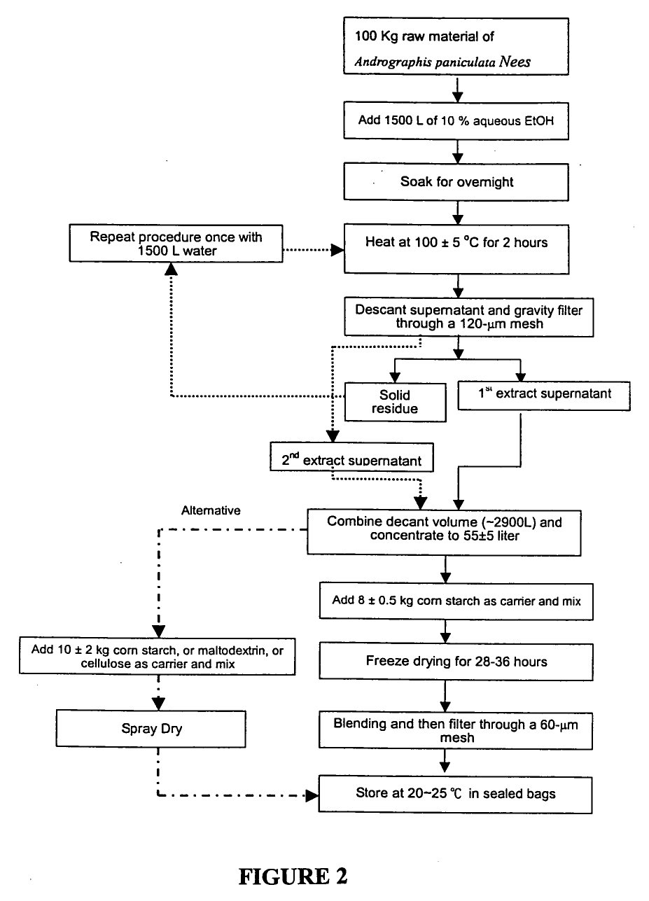

The herbal composition of the present invention was prepared as shown in the flowchart of FIG. 2. In particular, 100 kg of aerial parts of Andrographis paniculata Nees were added at room temperature to 1500 L de-ionized water containing 5 to 15% ethanol, and the mixture was heated at 100° C. for 2 hours. The obtained extract was then decanted through a 120 μm nylon mesh and stored in a 32,000 L stainless container. The extraction was repeated for the insoluble solid residues with additional 1500 L de-ionized water under the same conditions as described above. The second extract obtained was added to and pooled together with the first extract to form an extract with totally approximately 2950 L. After concentration under reduced pressure, the total volume of the extract was reduced to 50±5 L, and then 8.5±0.5 kg corn starch was added to said condensed extract and the mixture was well mixed. Freeze-drying was carried out at −25° C. for 36 hours at a pressure of 6 Kg/cm³. Or as an alternative, 10±2 kg corn starch or cellulose or maltodextrin could be added to the condensed extract and then the mixture was spray dried. The dry powder was ground and then filtrated through a 60-mesh filter and stored in sealed polyethylene bags at 20 to 25° C. Generally, 16±2 kg or 18±2 kg of the herbal composition could be obtained by the freeze-dry or spray-dry process. Standardization and evaluation of an extract of Andrographis paniculata Nees for pharmaceutical effectiveness may be achieved by subjecting Andrographis paniculata Nees to extraction to obtain an extract, and drying the extract (w/o carrier) to obtain a dry extract. In combination with appropriate analytical procedures, such as HPLC, the index or ingredient (e.g. AG) per gram of the dry extract of Andrographis paniculata Nees could be calculated, and then the amount needed to obtain expected efficacy could be determined. Normally, the final content of AG in the dry extract of Andrographis paniculata Nees was controlled at 2.5±0.5%.

### Example 3

### Screening on an Herbal Chip

A high-throughput method for discovering active ingredient(s) in herbs was already disclosed in U.S. Pat. No. 6,645,719. Its procedure is described as follows:

(a) Fractionation of Herb Extract with HPLC

Dried herb Andrographis paniculata Nees was ground to a fine powder and extracted by methanol, ethanol or distilled water, by a ratio of 50 grams of ground powder with 500 ml HPLC grade solvent at 25 to 30° C. The extraction was conducted with continuous blending for 5 min. by using a homogenizator (OMNI). Followed by centrifugation at 8,000 rpm (Beckman) for 30 min., the residue (pellet) was retreated twice as above. The clear supernatant of an herb extract was collected and then concentrated by a rotatory evaporator (Laborota 4000, HEIDOLPH) to a final volume of 30 ml. The concentrated extract was brought to 50 ml with a mixture of 50% water and 50% ethanol, and then mixed with a magnetic stir bar for 20 min. The extract was centrifuged at 8,000 rpm for 30 min. The clear supernatant was further centrifuged at 12,000 rpm for 10 min. and the resolution was conducted with HPLC (Shimadzu; TSK Gel ODS-80 column (4.6 mm×15 cm, 5 μm packing, Tosoh), a gradient of 0 to 100% ethanol with double distilled water for 96 minutes, flow rate 0.75 ml/min). Absorbance was monitored continuously at a wavelength of 205 nm. The HPLC eluates were then collected at 0.75 ml/min/fraction from time 0 to 96 minutes. The fractions containing the ingredients of Andrographis paniculata Nees were divided into three 96-well U-bottom microtiter plates by using an automated liquid handling system (MultiProbe II, Packard). The microtiter plates containing fractions of dried ingredients of Andrographis paniculata Nees were ready for storage or subjected to activity assay.

(b) Loading Samples onto the Coated Plastic Slides in Microarray Format

Microarrayer (BioGrid II, BioRobotic) was applied to spot fraction samples onto a coated plastic slide described in U.S. Pat. No. 6,645,719. The dried ingredients of Andrographis paniculata Nees in the microtiter plates were first dissolved and dispensed in 30% DMSO/0.1M carbonate buffer pH 9.5 (16 μl/well). A 4-pin (0.4 mm ID) tool was used to load samples from the 96-well microtiter plates onto the cavity chambers of the coated plastic slide. Biotin hydrazide with a concentration of 20 μg/ml was spotted onto the slides as a control. After the spots on the cavity chambers of the plastic slide were dried, the plastic slide was soak-treated with 1M ethanolamine (pH 8.0) at 37° C. for 2 hours.

(c) Signal Detection

Biotinylated tumor necrosis factor-alpha (B-TNFα) and Cy5-labeled strepavidin were used as probes for conducting hybridization. Two glass lids (22 mm×22 mm) were used to respectively cover the two cavity chambers of each plastic slide prior to loading 20 μl of B-TNF in TBST (0.5 μg/ml in 50 mM Tris-HCl, pH 7.3, 0.15 M NaCl, and 0.05% Tween 20) at 37° C. for 2 hours. The plastic slides were then washed 4 times by TBST, followed by drying the plastic slides at 37° C., covering the cavity chambers with glass lids as previously described. Twenty μl of Cy5-labeled strepavidin was loaded into each cavity chamber. The plastic slides were allowed to stand at 37° C. for 2 hours, followed by washing with TBST 4 times and then rinse with double distilled water 4 times. Finally, the plastic slides were dried at 37° C. and then scanned by the laser scanner (GenePix4000, Axon). The red fluorescent spots on the image showed that those fractions binds to B-TNFα.

### Example 4

### Evaluation of TNFα Antagonist Activity by In Vitro L929 Cell Proliferation/Cytotoxicity Assay

In vitro L929 cell proliferation/cytotoxicity assay was performed on microtiter plates. L929 cells were cultured in Eagle's Minimal Essential Medium (EMEM) containing 10% bovine serum, 1% P/S and 1% non-essential amino acids. Confluent L929 cells were washed with 2 ml PBS solution and then trypsinized, followed by resuspending in complete medium. Two hundards μl of cell suspension was aspirated for counting cell density and the other suspension was centrifuged at 1,500 rpm for 5 min. The supernatant was removed and the complete medium was added to the dilute cells to form a final concentration of 1.5×10⁵ cells/ml. One hundard μl of cell suspension was added to each well of 96-well flat-bottom microtitre plates and incubated for 24 hours in 5% CO₂ atmosphere at 37° C.

The test substance from Example 2 was resuspended in 1×PBS and sterilized with 0.22 μm filters. Various concentrations of a chemical were incubated for 1 hour with equal volume of commercial TNFα (0.2 ng/ml). Prior to the end of the 1 hour pre-incubation, the medium was removed from the 24 hour-incubated 96-well plate, followed by adding 50 μl fresh medium containing 4 μg/ml Actinomycin D (Act D). The 50 μl of pre-incubated mixture of the chemical and TNFα was transferred to the 96-well plate with the medium containing Act D to give the final concentration of 2 μg/ml Act D and TNFα 0.1 ng/ml. Act D 2 μg/ml and TNFα 0.1 ng/ml were added as positive control, and Act D 2 μg/ml was added as a negative control. After gently shaking for mixing, the 96-well plate was incubated for 24 hours in 5% CO₂ atmosphere at 37° C.

Cytotoxicity

The same sample as those for TNFα activity assay were added to a 96-well plate with the medium containing Act D to give the final concentration of 2 μg/ml Act D. The 96-well plate was gently shaken and incubated for 24 hours in 5% CO₂ atmosphere at 37° C. Then 50 μl XTT (2,3-bis(2-methoxy-4-nitro-5-sulfophenyl)-5-[(phenylamino)carbonyl]-2H-tetrazolium hydroxide) was added to each well, and then incubated for 4 hours. Read with ELISA reader at O.D 490/630 nm.

Calculation of the TNFα inhibition and cytotoxicity:

$$TNF α Inhibition \% = \frac{O . D {.}_{dilut + TNF + Act .} - O . D {.}_{TNF α + Act}}{O . D {.}_{Act only} - O . D {.}_{TNF α + Act}} \times 100 \%$$

$$Cytotoxicity \% = \frac{O . D {.}_{dilut . + Act D}}{O . D {.}_{Act D only}} \times 100 \%$$

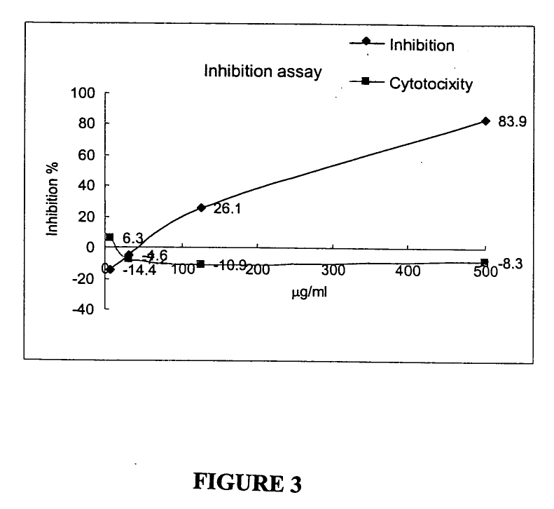

The effect of the herbal composition (see Example 2) on TNFα inhibitory activity was measured. The herbal composition showed 84% inhibitory activity at a concentration of 500 μg/ml, acting as a TNFα antagonist, and no cytotocixity was found (FIG. 3).

### Example 5

### Anti-Inflammatory Activity Test in Rats with Collagen-Induced Arthritis (a) Animal Preparation

SD rats with pathogen-free grade were kept in a climate-control environment for 4 days. The animals were sex- and age-matched, aged from 8 to 12 weeks. Collagen-induced arthritis was induced by immunization of rats with 300 μg of bovine native Type II collagen (CII, Chrondrex) in Incomplete Freund's Adjuvant (IFA, Sigma) emulsion, subcutaneously administered at the base of the tail, at a site of 2-3 cm. from body. Seven days after the initial immunization, immunity was boosted with 100 μg of CII. The rats were evaluated for disease three times a week and scored according to an established protocol measuring the paw volume.

(b) Treatment for Herbal Composition

Rats with arthritis were grouped into 6 groups, housed in polystyrene cages, each with 4-5 rats, containing wood shavings and fed standard rodent chow and water ad libitum. Daily intra-abdominal injection with 1 ml of the herbal composition containing 4.5 to 9 mg material prepared as described in Example 2; vehicle and dexamethasone were used as negative and positive controls. Treatment period was 7 days. The information of body weight and paw volumes, blood samples were collected at day 0, 3, 6, 10 and 14. Six days after the final dosing, all the animals were sacrificed. The affected hind limbs were removed for histological assessment. In the groups treated with the herbal composition there appeared a decreased percentage of the edema volumes for hind paws, which was about 48%. The decrease in edema maintained at 50% after treatment stopped, and lasted for at least 7 days until the study was terminated.

### Example 6

### In Vitro Safety Evaluation

To confirm possible adverse effects, extensive in vitro studies covering a broad range of physical function-related 67 molecular targets including G-protein coupled receptors, ion channel-coupled receptors, transporters, ion channels and enzymes, were conducted. The significant response criteria were determined as ≧50% inhibition or stimulation. Interactions found in the primary assay can be indicative of in vivo side effects or safety.

The results were summarized as in Table 1. None of the results met significance criteria for the herbal composition. It was revealed that andrographolide at a concentration of 25 μM in the herbal composition showed no significant side-effect.

TABLE 1
The significant response criteria were determined as ≧50% inhibition or
stimulation. No significant changes were observed in the assays.

| Assay | | |
|---|---|---|
| no | Target | % Inhibition |
| 1 | CYP450, 1A2 | 18 |
| 2 | CYP450, 2C19 | 45 |
| 3 | CYP450, 2C9 | 22 |
| 4 | CYP450, 2D6 | 9 |
| 5 | CYP450, 3A4 | 35 |
| 6 | Adenosine A₁ | −1 |
| 7 | Adenosine A_{2A} | 3 |
| 8 | Adenosine A₃ | 8 |
| 9 | Adrenergic α_{1A} | 1 |
| 10 | Adrenergic α_{1B} | −10 |
| 11 | Adrenergic α_{1D} | 5 |
| 12 | Adrenergic α_{2A} | 7 |
| 13 | Adrenergic β₁ | −18 |
| 14 | Adrenergic β₂ | −4 |
| 15 | Adrenergic β₁ | −3 |
| 16 | Bradykinin B₁ | −3 |
| 17 | Bradykinin B₂ | −3 |
| 18 | Calcium | −6 |
| | Channel | |
| | L-Type, | |
| | Benzothiazepine | |
| 19 | Calcium | −12 |
| | Channel | |
| | L-Type, | |
| | Dihydropyridine | |
| 20 | Calcium | −1 |
| | Channel | |
| | N-Type | |
| 21 | Dopamine D₁ | 2 |
| 22 | Dopamine D₂₅ | 0 |
| 23 | Dopamine D₃ | 5 |
| 24 | Dopamine D_{4,2} | 4 |
| 25 | Endothelin ET_{A} | 3 |
| 26 | Endothelin ET_{B} | 5 |
| 27 | Epidermal | 2 |
| | Growth Factor | |
| | (EGF) | |
| 28 | Estrogen ERα | 1 |
| 29 | GABA_{A}, | 15 |
| | Agonist Site | |
| 30 | GABA_{A}, | 4 |
| | Benzodiazepine, | |
| | Central | |
| 31 | GABA_{B}, | 3 |
| | Non-Selective | |
| 32 | Glucocorticoid | 6 |
| 33 | Glutamate, | 13 |
| | Kainate | |
| 34 | Glutamate, | 7 |
| | NMDA, | |
| | Agonism | |
| 35 | Glutamate, | 1 |
| | NMDA, | |
| | Glycine | |
| 36 | Glutamate, | 11 |
| | NMDA, | |
| | Phencyclidine | |
| 37 | Histamine H₁ | 1 |
| 38 | Histamine H₂ | −6 |
| 39 | Histamine H₃ | 3 |
| 40 | Imidazoline I₁, | −7 |
| | Central | |
| 41 | Interleukin IL-1 | −11 |
| 42 | Leukotriene, | −1 |
| | Cysteinyl | |
| | CysLT₁ | |
| 43 | Muscarinic M₁ | 12 |
| 44 | Muscarinic M₂ | −7 |
| 45 | Muscarinic M₃ | 0 |
| 46 | Neuropeptide | −13 |
| | Y₁ | |
| 47 | Neuropeptide | −3 |
| | Y₂ | |
| 48 | Nicotinic | 3 |
| | Acetylcholine | |
| 49 | Opiate δ (OP1, | −5 |
| | DOP) | |
| 50 | Opiate κ (OP2, | −1 |
| | KOP) | |
| 51 | Opiate μ (OP3, | −3 |
| | MOP) | |
| 52 | Phorbol Ester | 5 |
| 53 | Platelet | −11 |
| | Activating | |
| | Factor (PAF) | |
| 54 | Potassium | 9 |
| | Channel [K_{ATP}] | |
| 55 | Potassium | 27 |
| | Channel HERG | |
| 56 | Purinergic P₂ₓ | 5 |
| 57 | Purinergic P_{2Y} | 29 |
| 58 | Serotonin(5-Hydroxytryptamine) | 5 |
| | 5-HT_{1A} | |
| 59 | Serotonin(5-Hydroxytryptamine) | 2 |
| | 5-HT₃ | |
| 60 | Sigma δ₁ | 3 |
| 61 | Sigma δ₂ | 4 |
| 62 | Sodium | 11 |
| | Channel, Site 2 | |
| 63 | Tachykinin NK₁ | −3 |
| 64 | Testosterone | 7 |
| 65 | Transporter, | −3 |
| | Dopamine | |
| | (DAT) | |
| 66 | Transporter, | −11 |
| | GABA | |
| 67 | Transporter, | 2 |
| | Norepinephrine | |
| | (NET) | |
| 68 | Transporter, | −5 |
| | Serotonin(5-Hydroxytryptamine) | |
| | (SERT) | |

### Example 7

### In Vivo Adverse Effect Study

To further evaluate the safety of the herbal composition, a serial pharmacology studies with regards to central nerve system, cardiovascular system, respiratory system and renal function were conducted as follows. The dosage for all of the studies was 666 mg/kg (with an equivalence of andrographolide 14 mg/kg).

(a) Prolongation in Bleeding Time in Mice

Test substance was administrated P.O. to a group of preselected CD-1 derived male or female mice weighing 22±2 g at one hour before standardized transaction of the tip (0.3 mm) of the tail. The mice, in restraining holders, were immediately suspended vertically with the distal 2 cm of each tail immersed in a test tube containing saline at 37° C. A maximum cut-off time of 180 seconds observation period, the measurement would be discontinued, and any subsequent bleeding would not be considered. Prolongation of bleeding time by 50% or more relative to the control group is considered significant.

(b) Autonomic Signs, Behavior and Mortality in Mice

The herbal composition was administrated orally to a group of CD-1 derived male or female mice weighing 22±2 g, and the animals were observed for the presence of acute toxic symptoms (mortality, convulsions, tremors, muscle relaxation, sedation etc.) and autonomic effects (diarrhea, salivation, lacrimation, vasodilation, piloerection . . . etc.) during the first hour. Mortality was also subsequently noted 3, 24, 48 and 72 hours after treatment.

(c) Changes in Body Temperature in Mice

The herbal composition was administrated orally to a group of CD-1 derived male or female mice weighing 22±2 g, and rectal temperature was measured with a Monitoring Thermometer (Thermalert, TH-5) immediately before (0 minute) and at 30, 60 and 120 minutes post dosing. An averaged percent change in temperature by 5% or more (≧5%) at any post-treatment time interval in relative to the pre-treatment (0 minute) time is considered significant.

(d) Behavioral Depression in Mice

The herbal composition was administrated orally to a group of CD-1 derived male or female mice weighing 22±2 g. After one four, ten parameters were measured: ataxia, exploration, palpebral size, pinna reflex, placing, reactivity, righting reflex, spontaneous activity, startle response, touch response. Each parameter scored 2 points for normally yielding a total of 20 points×5 mice=100 points. A score of 65 or less indicates behavioral depression.

(e) Motor Coordination in Mice

The herbal composition was administrated P.O. to a group of preselected CD-1 derived male or female mice weighing 22±2 g at one hour before being placed on a turning rod. Selected animals were those which remained on 115 rpm turning rod covered with roughened tubing of 3.5 cm diameter for more than 1 minute. Motor incoordination was then indicated, if 50% or more of the tested animals exhibited incapability to remain on the turning rod for 1 minute.

(f) Motor Stimulation in Mice

The herbal composition was administrated P.O. to a group of preselected CD-1 derived male or female mice weighing 22±2 g. After one hour, ten parameters were measured: irritability, hyperactivity, increased palpebral size, increased startle response, increase sensitivity of touch, increased exploration, piloerection, Straub tail, tremors, and convulsions. Maximum abnormal condition for each observation scores 2 points yielding a total of 2×10 parameters×5 mice=100 points. A score of 20 points or greater indicates significant stimulation.

(g) Rate and Depth of Respiration in Mice

The herbal composition was administrated P.O. to a group of preselected CD-1 derived male or female mice weighing 22±2 g at one hour before being placed in a 1 L beaker for observation. Change in respiration for each animal in relative to vehicle-treated controls was scored as plus for increase in rate with decrease in depth and as negative for decrease in rate with increase in depth. A positive or negative exhibition in 50% or more of tested animals was considered significant.

(h) Mean Arterial Blood Pressure at Resting or After Tilting in Rats

Groups of 5 Wistar derived male rats weighing 250±20 g were employed. Each animal was secured supine upon a tilt board in a horizontal position under Propofol anesthesia (15 mg/5 ml/kg, i.v.). The tail artery was cannulated with polyethylene (PE160) catheter connected via a Statham (P23XL) pressure transducer to NEC/San-Ei amplifier and the data acquisition and analysis system (power lab 4/20). Upon recovery from anethesthia (2-3 hours later), the herbal composition or vehicle was administrated orally. Before (0 minute) and 60 minutes post dosing, mean arterial blood pressure was recorded and each animal was than tilted 90° C., in approximately one second, to a vertical position for 2 minutes. The lowest mean blood pressure value during the 2 minute period was recorded. A tilt response-induced reduction ≧10% of the mean arterial blood pressure relative to values at the horizontal position indicates significant activity. The reduction ≧10% of the mean arterial blood pressure between 60 minute post-dosing and 0 minute (before dosing) was also evaluated.

(i) Mydriasis in Mice

The herbal composition was administrated P.O. to a group of preselected CD-1 derived male or female mice weighing 22±2 g. Immediately (0 time) before and 60 minutes after dosing, pupil diameter was measured under constant bright light by means of dissecting microscope with micrometer eye piece. If pupil diameter (normally ≦0.3 mm) measures are equal to or greater than 1 mm, α-adrenoreceptor agonism, ganglionic blockade or peripheral anticholinergic activity was indicated.

(j) Serum Total Level of Cholesterol, Triglycerides and HDL

The herbal composition was administrated P.O. to a group of preselected CD-1 derived male or female mice weighing 22±2 g. After 24 hours and overnight fasting (about 16 hours) with water ad libitum, serum was obtained for measuring lipoprotein (HDL) and TG by an enzymatic method (Automatic analyzer model 7050 and Wako cholesterol & HDL and TG diangnostic kit). A change of 20% or more in total HDL or LDL and 40% or more in TG relative to the vehicle-treated group was considered significant.

(k) Hepatotoxicity in Mice

The herbal composition was administrated P.O. to a group of preselected CD-1 derived male or female mice weighing 22±2 g. After 24 hours and fasting overnight (about 16 hours) with water ad libitum, alanine aminotransferase activity was determined for each animal by an optimized UV method (Automatic analyzer model 7050 and Wako ALT diagnostic kit). A 100% or more increase (≧200% of control) in ALT relative to the vehicle control group was considered significant.

(l) Renal Function in Fasted Rats

Groups of 5 Wistar derived male or female overnight fasted rats weighing 150±20 g were used. Each animal was placed in individual cage and hydrated with physiological saline (15 ml/kg) administrated orally together with the herbal composition or vehicle. Urine volume was measured over the ensuing 6-hour period and then urine was centrifuged (×400 g, 10 minutes) and assayed for sodium and potassium concentration with an automatic electrolyte analyzer (Cibacorning, Model 644). The urinary electrolyte excretion is expressed as μEq/100 g body weight. A 100% or more increase (≧200% control) in sodium excretion (Natriuresis) or potassium excretion (Kaliuresis), or 50% or more increase (≧150% control) in urine volume output of treated versus control animals was considered significant. A 50% or more decrease (≦50% control) in sodium excretion, potassium excretion, or urine volume output in treated versus control animals was considered significant.

(m) Serum Glucose in Fasted Mice

Male or female CD-1 derived mice weighing 22±2 g were fasted overnight prior to study. In a group of five animals, the herbal composition was administrated orally and blood samples were obtained 90 minutes later. A change in serum glucose by 20% or more relative to vehicle-treated control animals indicates significance activity.

(n) Gastric Acidity in Fasted Rats

A group of 5 Wistar derived male or female rats weighing 150±20 g was fasted overnight. Two hours before and 1 hour after oral administration of the herbal composition, gastric acidity (μEq HCL/ml) was determined following orally administered with normal saline (2 ml). The percent increase was calculated by comparison between pre-treatment and post-treatment values. A 50% or more changes in gastric acidity was considered significant.

(o) Gastric Irritation in Fasted Rats

A group of 5 Wistar derived male or female rats weighing 150±20 g was fasted overnight. The herbal composition was administrated orally. The animals were sacrificed 4 hours later and gastric irritation/ulceration was scored according to an arbitrary system: 0=no lesions, 1=hyperemia, 2=one or two slight lesions, 3=more than 2 slight lesions or serve lesions, 4=very serve lesions. Test substance-induced ulceration of 50% or more relative to the control Aspirin (150 mg/Kg, PO) response was considered significant.

(p) Gastrointestinal Motility in Fasted Mice

Groups of CD-1 derived male or female mice weighing 25±2 g were fasted with free access to water for 16 hours prior to testing. The herbal composition was administrated orally. At 60 minutes post dosing, the animals were given a suspension of 5% charcoal in 10% Arabic solution (0.3 ml/animal, p.o.) and sacrificed 15 minutes later. The intestine was removed and the length of the gut in cm (GL) as well as distance of charcoal movement from the pylorus to the front of the charcoal column in cm (CP) was measured. Intestinal transit (IT) is calculated as IT=(CP/GL)×100%. A 30% or more increase or a decrease in intestinal transit (IT) relative to the vehicle-treated group was considered significant.

As the results showed in Table 2, oral administration of the herbal composition at 666 mg/kg did not cause any significant changes, in accordance with the established criteria in the above mouse and rat assays.

TABLE 2

| | Study | Significance | +/− |
|---|---|---|---|
| (a) | Prolongation in bleeding time in mice | N | +38% |
| (b) | Autonomic signs, behavior | N | none |
| | and mortality in mice | | |
| (c) | Changes in body temperature in mice | N | none |
| (d) | Behavioral depression in mice | N | none |
| (e) | Motor coordination in mice | N | none |
| (f) | Motor stimulation in mice | N | none |
| (g) | Rate and depth of respiration in mice | N | none |
| (h) | Mean arterial blood pressure at | N | none |
| | resting or after tilting in rats | | |
| (i) | Mydriasis in mice | N | none |
| (j) | Serum total level of cholesterol, | N | HDL: −2% |
| | triglycerides and HDL | | LDL: +2% |
| | | | TG: +5% |
| (k) | Hepatotoxicity in mice | N | −1.0% |
| (l) | Renal function in fasted rats | N | Urine vol: |
| | | | −44% |
| | | | Na: +11% |
| | | | K: +69% |
| (m) | Serum glucose in fasted mice | N | +18% |
| (n) | Gastric acidity in fasted rats | N | +20% |
| (o) | Gastric irritation in fasted rats | N | +11% |
| (p) | Gastrointestinal motility in | N | +16% |
| | fasted mice | | |

Note:
Changes in increase are indicated as “+”, and changes in decrease are indicated as “−”.
No changes or not observed are indicated as “none”.

### Example 8

### Preliminary Open Clinical Investigation on Effect of the Herbal Composition in Human

The objective of the study is to evaluate efficacy and safety of the herbal composition in patients with osteoarthritis (OA), rheumatoid arthritis (RA), psoriasis and neuralgia caused by ankylosing spondylitis (AS).

Study Plan

No. of patients: 34 patients were selected for the study

Age group: 40-75 years old

Inclusion/exclusion criteria: Random selection and diagnosed with the diseases by clinical doctors. Both sexes are included. Patients with complicated chronic diseases like diabetes, tuberculosis or pregnant or in chemotherapy were excluded from the study.

Dose: 2 to 6 capsules/day, 420±15 mg/capsule prepared from Example 2

Duration: 7 to 14 days

Parameters used for assessment: Subjective scoring in all patients for symptomatic relief.

Results

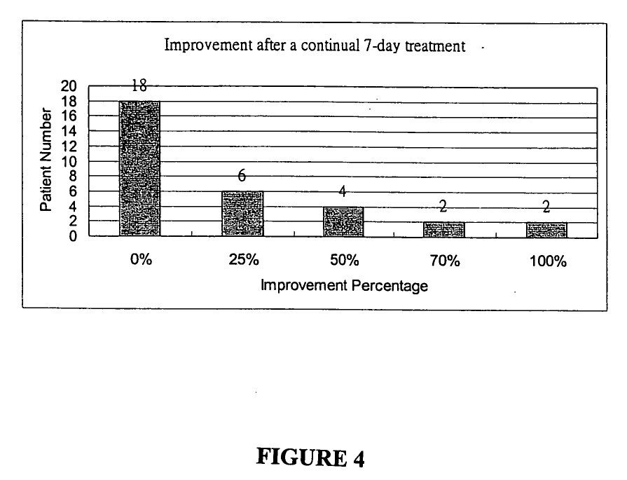

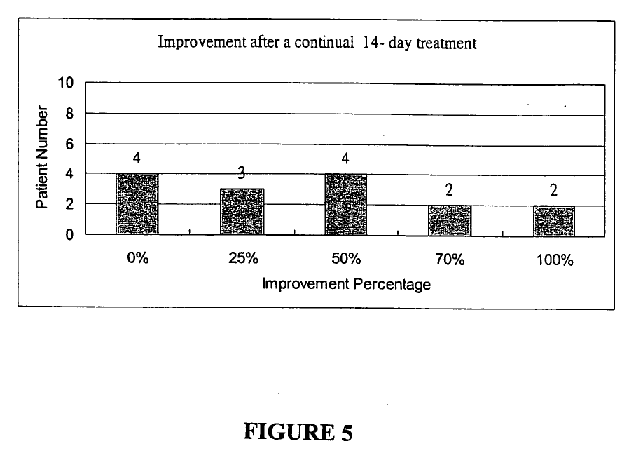

There were 2 patients with RA, 5 patients with OA, 2 patients with psoriasis, and 25 patients with neuralgia in this study. The improvement of psoriatic patients is not analyzed in the following evaluation (1) and (2).
(1) Evaluation of systemic symptomatic relief in all of the patients after a 7-day continual administration (FIG. 4):
   2 patients out of 32 (6%) showed 100% improvement;
   2 patients out of 32 (6%) showed 70% improvement;
   4 patients out of 32 (13%) showed 50% improvement;
   6 patients out of 32 (19%) showed 25% improvement; and
   18 patients out of 32 (13%) showed no improvement.
(2) Evaluation of systemic symptomatic relief in all of the patients after a 14-day continual administration (FIG. 5), wherein there are 15 patients out of the 32 patients carried on the treatment, and all of the 15 patients are evaluated:
   2 patients out of 15 (13%) showed 100% improvement;
   2 patients out of 15 (13%) showed 70% improvement;
   4 patients out of 15 (27%) showed 50% improvement;
   3 patients out of 15 (20%) showed 25% improvement; and
   4 patients out of 15 (27%) showed no improvement.

The results are shown in Table 3.

TABLE 3

| | Improvement % | | | | |
|---|---|---|---|---|---|
| | 0% | 25% | 50% | 70% | 100% |
| | N = 2 (Discontinue after | | | | |
| RA | 7 days: 2 patients) | | | | |
| Day 7 | 0 | 1 | 0 | 0 | 1 |
| Day 14 | NA | NA | NA | NA | NA |
| | N = 5 (Discontinue after | | | | |
| OA | 7 days: 1 patients) | | | | |
| Days 7 | 4 | 0 | 0 | 1 | 0 |
| Day 14 | 2 | 0 | 1* | 1 | 0 |
| | N = 25 (Discontinue | | | | |
| Neuralgia | after 7 days: 2 patients) | | | | |
| Day 7 | 14 | 5 | 4 | 1 | 1 |
| Day 14 | 2 | 3 | 3 | 1 | 2 |

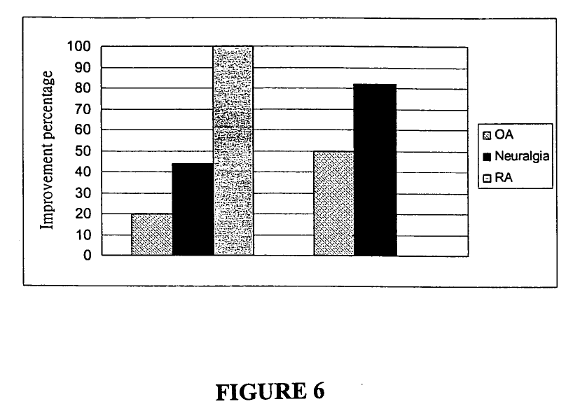

All the 32 patients had no chief complaints during the study. Among the 2 patients with RA, 5 patients with OA and 25 patients with neuralgia, the improvement ≧25% in symptomatic relief in different patient groups are shown in FIG. 6.

After continual 7-day administration,
2 RA patients out of 2 patients showed improvement (100%);
1 OA patients out of 5 patients showed improvement (20%); and
11 neuralgia patients out of 25 patients showed improvement (44%).

After continual 14-day administration among 15 patients,
2 OA patients out of 4 patients showed improvement (50%); and
9 neuralgia patients out of 11 patients showed improvement (82%).

Similar results were achieved in the enrolled 2 patients with moderate-to-servere psoriasis. After 14-day continual administration, both patients experienced 50-70% improvement from baseline.

What is claimed is:

1. A method for treating arthritic disorders, skin inflammatory disorders and pain, comprising administering to a subject an extract of Andrographis paniculata Nees.

2. The method of claim 1, wherein the administration is oral, intravenous, parenteral or transdermal administration.

3. The method of claim 1, wherein the extract of Andrographis paniculata Nees binds to TNFα and inhibits the activity of TNFα.

4. The method of any one of claims 1 to 3, wherein the arthritic disorder is arthritis or neuralgia induced by ankylosing spondylitis.

5. The method of claim 4, wherein the arthritis is osteoarthritis or rheumatoid arthritis.

6. The method of any one of claims 1 to 3, wherein the skin inflammatory disorders are psoriasis, eczema and dermatitis.

7. The method of any one of claims 1 to 3, wherein the extract of Andrographis paniculata Nees is an ethanol in water extract containing 1 to 3% by weight of andrographolide.

8. The method of any one of claims 1 to 3, wherein the extract of Andrographis paniculata Nees is formulated to a powder, capsule, tablet, solution, syrup, solution, suspension, emulsion, ointments, creams, lotions or patch.

9. The method of any one of claims 1 to 3, wherein the extract of Andrographis paniculata Nees is administered in an amount of 300 to 3200 mg/day, preferably 400 to 2000 mg/day.

10. An herbal composition for treating arthritic disorders, skin inflammatory disorders and pain, comprising an extract of Andrographis paniculata Nees and a pharmaceutically acceptable excipient.

11. The herbal composition of claim 10, wherein the extract of Andrographis paniculata Nees binds to TNFα and inhibits the activity of TNFα.

12. The herbal composition of claim 10 or 11, wherein the arthritic disorder is arthritis or neuralgia induced by ankylosing spondylitis.

13. The herbal composition of claim 12, wherein the arthritis is osteoarthritis or rheumatoid arthritis.

14. The herbal composition of claim 10 or 11, wherein the skin inflammatory disorders are psoriasis, eczema and dermatitis.

15. The herbal composition of claim 10 or 11, wherein the extract of Andrographis paniculata Nees is an ethanol in water extract containing 1 to 3% by weight of andrographolide.

16. The herbal composition of claim 10 or 11, which is formulated to a powder, capsule, tablet, liquid or syrup.
